(19) United States
(12) Patent Application Publication
(10) Pub. No.: US 2026/0096937 A1
(43) Pub. Date: Apr. 9, 2026

(54) HYGIENE PRODUCT PROVIDING DEVICE

(71) Applicant: SHARP KABUSHIKI KAISHA, Sakai City (JP)

(72) Inventors: Kentaro KITASUMI, Sakai City (JP); Takeshi FUJIWARA, Sakai City (JP); Taiyo SEKIGUCHI, Sakai City (JP); Hiroaki GOI, Sakai City (JP); MOTOKI TANIMURA, Sakai City (JP)

(21) Appl. No.: 19/352,625

(22) Filed: Oct. 8, 2025

(30) Foreign Application Priority Data
Oct. 9, 2024 (JP) 2024-177203

(51) Int. Cl. A61F 15/00

(52) U.S. Cl. CPC A61F 15/00

(57) ABSTRACT

A hygiene product providing device includes: a housing configured to accommodate a plurality of hygiene products in a stacked manner; a takeout port of the hygiene products disposed below a front surface portion of the housing; and a shutter configured to open and close the takeout port. When the shutter is opened, one hygiene product located at the lowermost portion in the housing is exposed from the takeout port, and a hygiene product located above the one hygiene product located at the lowermost portion is not exposed from the takeout port.

### BACKGROUND OF THE INVENTION

### 1. Technical Field

The present disclosure relates to a technique of a hygiene product providing device for providing a hygiene product such as a sanitary product.

### 2. Description of the Related Art

Conventionally, hygiene product providing devices for providing a hygiene product such as a sanitary product have been known. For example, WO 2023/148978 A discloses a hygiene product providing dispenser for providing a hygiene product to a user, the hygiene product providing dispenser including: a storage part for storing the hygiene product; a discharge part for discharging the hygiene product stored in the storage part from the storage part; and a control part for operating the discharge part so as to discharge the hygiene product when there is a request for the provision of the hygiene product from the user if the request is appropriate.

### SUMMARY OF THE INVENTION

An object of the present disclosure is to provide a more convenient hygiene product providing device capable of appropriately providing one hygiene product.

A hygiene product providing device according to one aspect of the present disclosure includes: a housing configured to accommodate a plurality of hygiene products in a stacked manner; a takeout port of the hygiene product disposed below a front surface portion of the housing; and a shutter configured to open and close the takeout port. In the hygiene product providing device, when the shutter is opened, one hygiene product located at the lowermost portion in the housing is exposed from the takeout port, and a hygiene product located above the one hygiene product located at the lowermost portion is not exposed from the takeout port.

According to the hygiene product providing device according to one aspect of the present disclosure, one hygiene product can be appropriately provided.

### BRIEF DESCRIPTION OF THE DRAWINGS

FIG. 1 is a perspective view showing an external configuration of a hygiene product providing device according to one embodiment;

FIG. 2 is a front view of the hygiene product providing device shown in FIG. 1;

FIG. 3 is a perspective view showing a state in which a shutter of the hygiene product providing device shown in FIG. 1 is opened;

FIG. 4 is a front view showing a state in which a shutter of the hygiene product providing device shown in FIG. 2 is opened;

FIG. 5 is a perspective view showing a state where a front surface member of the hygiene product providing device shown in FIG. 3 is removed;

FIG. 6 is a perspective view showing a configuration around a partition member and a bottom plate provided in the hygiene product providing device;

FIG. 7 is a side view showing a configuration around the partition member and the bottom plate in a state where thin sanitary products are accommodated;

FIG. 8 is a perspective view showing a configuration around the partition member and the bottom plate in a state where the thin sanitary products are accommodated;

FIG. 9 is a side view showing a configuration around the partition member and the bottom plate in a state where thick sanitary products are accommodated;

FIG. 10 is a perspective view showing a configuration around the partition member and the bottom plate in a state where the thick sanitary products are accommodated;

FIG. 11 is a side view for explaining the operation of the shutter and the partition member in a state where the thin sanitary products are accommodated;

FIG. 12 is a side view for explaining the operation of the shutter and the partition member in a state where the thin sanitary products are accommodated;

FIG. 13 is a side view for explaining the operation of the shutter and the partition member in a state where the thin sanitary product is accommodated;

FIG. 14 is a side view for explaining the operation of the shutter and the partition member in a state where the thin sanitary product is accommodated;

FIG. 15 is a perspective view illustrating an operation mechanism when the shutter and the partition member are in a state illustrated in FIG. 7;

FIG. 16 is a perspective view illustrating the operation mechanism when the shutter and the partition member are in a state illustrated in FIG. 11;

FIG. 17 is a perspective view illustrating the operation mechanism when the shutter and the partition member are in a state illustrated in FIG. 12;

FIG. 18 is a perspective view illustrating the operation mechanism when the shutter and the partition member are in a state illustrated in FIG. 13; and

FIG. 19 is a perspective view illustrating the operation mechanism when the shutter and the partition member are in a state illustrated in FIG. 14.

### DETAILED DESCRIPTION

Hereinafter, an embodiment of the present disclosure will be described with reference to the drawings. In the following description, the same components are denoted by the same reference numerals. Their names and functions are the same. Therefore, detailed descriptions thereof will not be repeated.

In the present embodiment, a hygiene product providing device for providing hygiene products such as sanitary products, disposable diapers, and tissue paper will be described. More specifically, a hygiene product providing device 1 suitable for providing a sanitary product which is an example of a hygiene product will be described. The hygiene product providing device 1 is installed, for example, in a private room of a toilet room or near a wash basin in a facility (for example, a public facility, a restaurant, a shopping center, a hotel, a station, or the like) used by an unspecified large number of people, and can provide sanitary products to a user (user) in the toilet room.

### Overall Configuration of Hygiene Product Providing Device

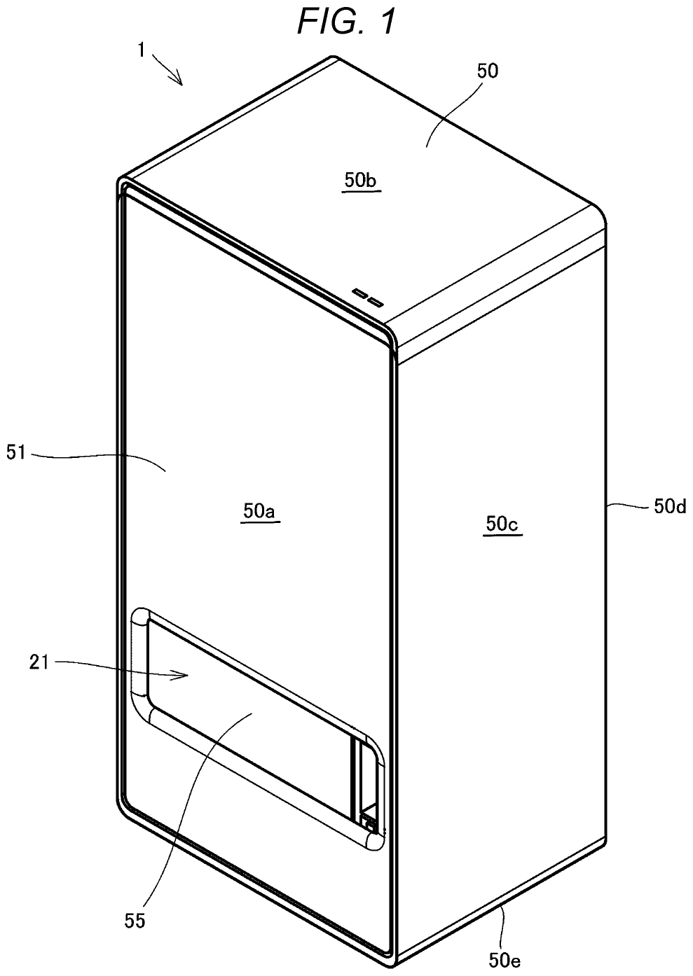

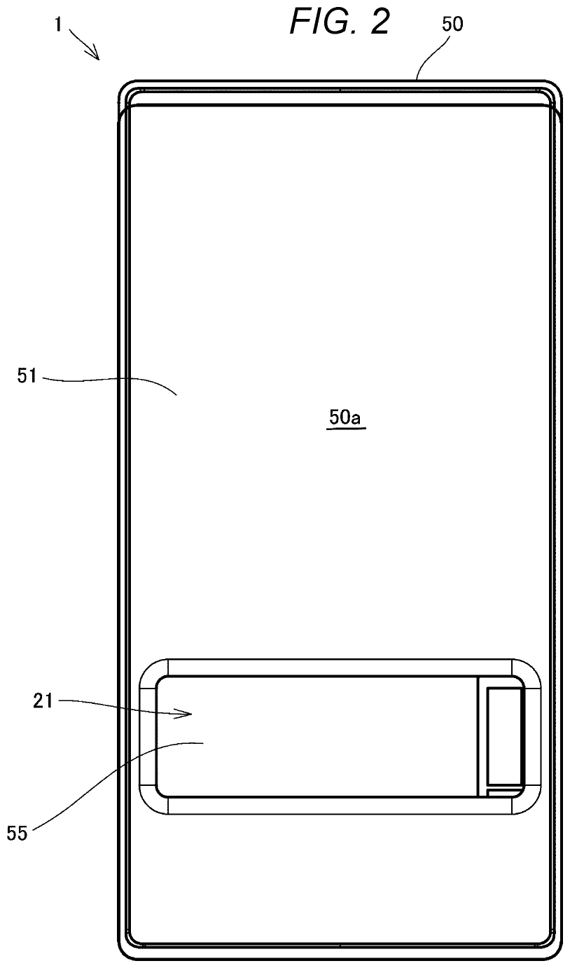

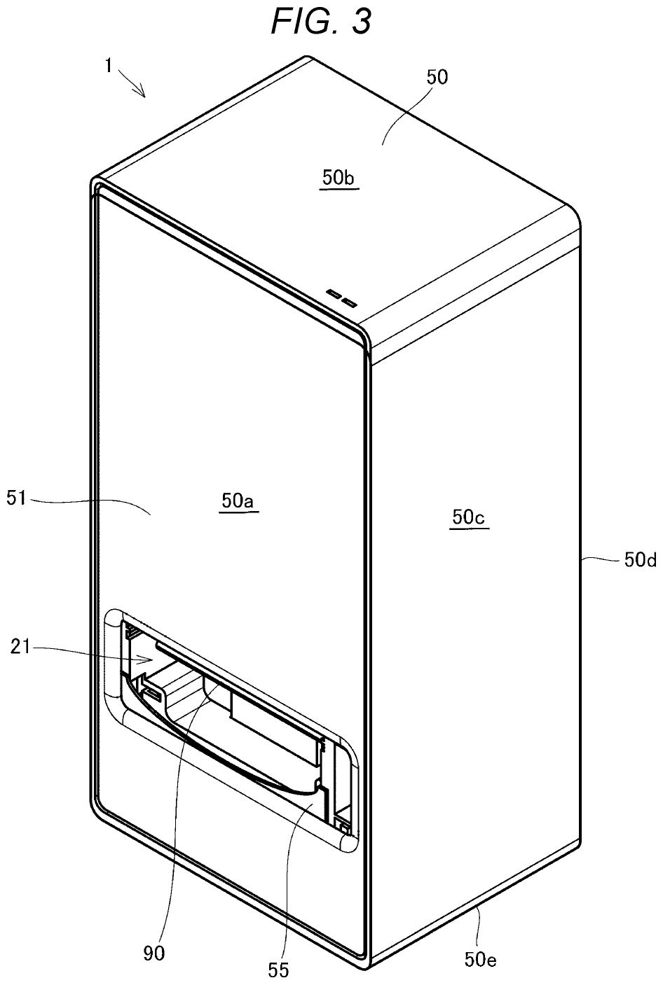

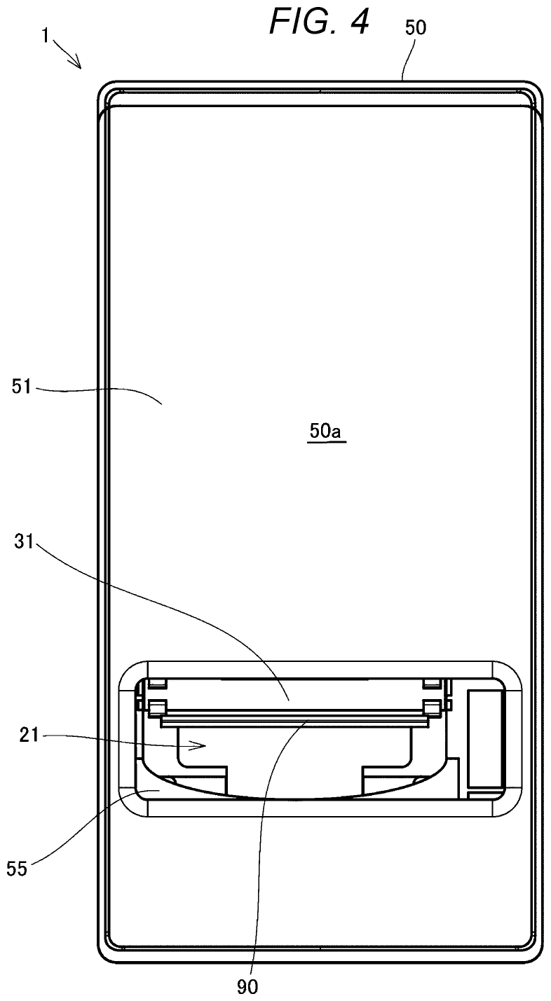

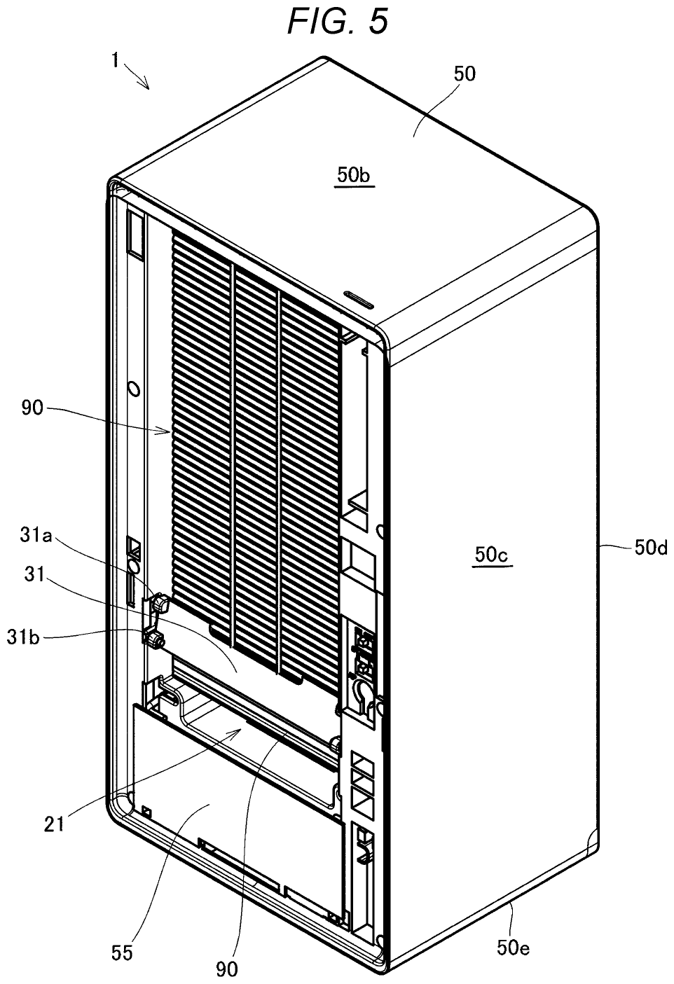

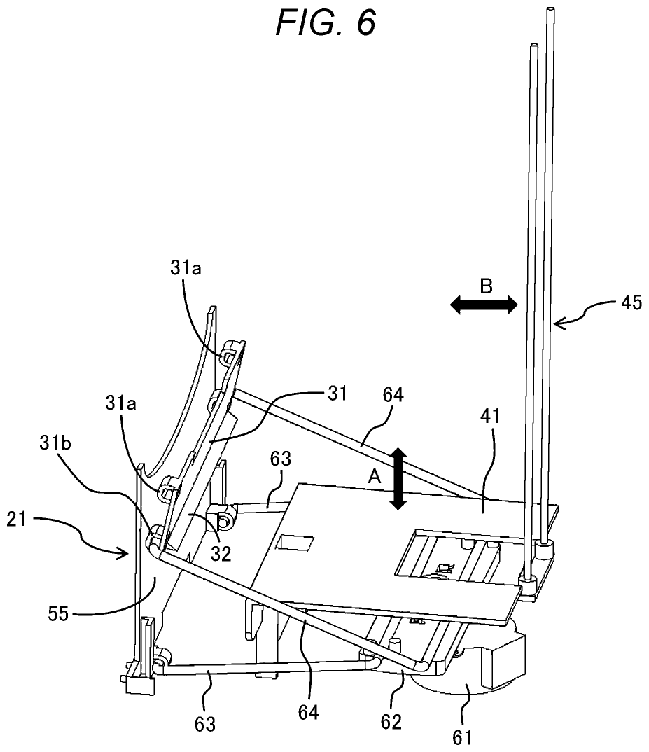

First, an overall configuration of a hygiene product providing device 1 (hereinafter, it is also simply referred to as a providing device 1) according to the present embodiment will be described. FIGS. 1 to 4 illustrate an appearance of the providing device 1. FIGS. 1 and 2 illustrate a state in which a shutter 55 of a sanitary product takeout port 21 is closed. FIGS. 3 and 4 illustrate a state in which the shutter 55 of the takeout port 21 is opened. FIG. 5 illustrates an internal configuration of a housing 50 in a state where a front surface member 51 is removed. FIG. 6 illustrates a partition member 31 and a bottom plate 41 provided inside the providing device 1 and the peripheral configuration thereof.

The outer shape of the providing device 1 is mainly formed of the housing 50 having a substantially rectangular parallelepiped shape. The housing 50 has a front surface portion 50a, an upper surface portion 50b, a side surface portion 50c, a back surface portion 50d, and a bottom surface portion 50e. In the present embodiment, the front surface portion 50a is formed of a front surface member 51, and the back surface portion is formed of a back surface member (not illustrated). The front surface member 51 and the back surface member are configured to be detachable from the main body portion of the housing 50 forming the upper surface portion 50b, the left side surface portion 50c, the right side surface portion 50c, and the like.

In the housing 50, a plurality of sanitary products 90, 90, . . . are accommodated in a stacked state (see FIG. 5). The providing device 1 according to the present embodiment can provide sanitary products of different sizes, but the sizes of a plurality of sanitary products accommodated at a time in the housing 50 are preferably the same.

The takeout port 21 of the sanitary product 90 is provided in a lower portion of the front surface portion 50a of the housing 50. The takeout port 21 is formed by opening a part of the front surface member 51.

Here, the lower portion of the front surface portion 50a means a lower side than the central position in the height direction of the housing 50. For example, the takeout port 21 is preferably provided at a position within 20 cm from the bottom surface portion 50c of the housing 50. In addition, the takeout port 21 is provided so as to correspond to the position of the lowermost sanitary product 90a among the plurality of sanitary products 90 accommodated in the housing 50.

A partition member (plate-like member) 31 is provided behind the front surface member 51 inside the housing 50 (see FIG. 5). The partition member 31 is located above the takeout port 21. When the shutter 55 is opened, the partition member 31 is inserted from the front between one sanitary product 90a located at the lowermost portion in the housing 50 and the sanitary product located at the second lowermost portion. As a result, one sanitary product 90 located at the lowermost portion in the housing 50 can be exposed from the takeout port, and the sanitary product 90 located above the sanitary product 90 located at the lowermost portion can be prevented from being exposed from the takeout port 21 (see FIG. 4 and the like).

As shown in FIG. 4, in a state where the shutter 55 is opened, the second sanitary product 90 from the bottom and some sanitary products 90 located above the second sanitary product are hidden by the partition member 31. In addition, since the sanitary product 90 located at a higher position in the housing 50 is located above the opening position of the takeout port 21, the sanitary product is in a state of being shielded by the front surface member 51 of the housing 50. As a result, the user can take out only one sanitary product 90 exposed from the takeout port 21, but cannot touch the other sanitary product 90 disposed in the housing 50.

The takeout port 21 is provided with the shutter 55 that shields the takeout port 21. The shutter 55 is configured to be movable in the vertical direction by a motor 61 provided in the providing device 1. For example, the shutter 55 moves in the vertical direction according to a detection result of a human sensor (not illustrated) disposed in the vicinity of the takeout port 21 to open and close the takeout port 21.

Meanwhile, hygiene products such as sanitary products and disposable diapers vary in individual sizes (for example, thickness, length and width) depending on manufacturers and respective products. For example, the thickness of sanitary products is greatly different between a manufacturer unit and a product unit depending on applications, service life, and the like. If it is possible to provide a plurality of types of sanitary products having different thicknesses, the convenience of the providing device 1 is improved.

Since the providing device 1 according to the present embodiment includes the bottom plate 41 and a rear support member 45 having the following configurations, it is possible to provide sanitary products having different sizes.

In the housing 50, the bottom plate 41 on which the sanitary product 90 is placed is provided. The bottom plate 41 is configured to be vertically movable in accordance with the size (specifically, the thickness of the sanitary product 90) of the sanitary product 90 (see an arrow A in FIG. 6).

The rear support member 45 for supporting the sanitary product 90 accommodated in the housing 50 is provided on the rear side in the housing 50. The rear support member 45 extends in the vertical direction at the rear in the housing 50. In the present embodiment, the position of the rear support member 45 in the front-rear direction is configured to be movable (see an arrow B in FIG. 6).

Since the bottom plate 41 which is vertically movable and the rear support member 45 which is movable back and forth are provided, the providing device 1 can cope with providing sanitary products of various sizes. For example, since the providing device 1 includes the bottom plate 41 that is vertically movable, the bottom plate 41 can be set to an appropriate height according to the thickness of the sanitary product 90 accommodated in the housing 50. In addition, for example, since the providing device 1 includes the rear support member 45 capable of moving back and forth, the rear support member 45 can be installed at an appropriate position according to the length (depth dimension) in the front-back direction of the sanitary product 90 accommodated in the housing 50.

In one example, the vertical position (height) of the bottom plate 41 is manually changed. When the height of the bottom plate 41 is manually changed, for example, the height of the bottom plate 41 can be adjusted by fixing the bottom plate 41 on the base using a screw or the like whose height can be adjusted. In another example, the height of the bottom plate 41 can be automatically changed using a motor or the like.

In one example, the position of the rear support member 45 in the front-rear direction is manually changed. In another example, the position of the rear support member 45 can be automatically changed using a motor or the like.

When the positions of the bottom plate 41 and the rear support member 45 are manually changed, the positions of the bottom plate 41 and the rear support member 45 are determined in advance for each type of sanitary products, and when the type of sanitary products to be accommodated in the housing 50 is determined, a worker may move the positions of the bottom plate 41 and the rear support member 45 to the set positions. Then, in a state where the bottom plate 41 and the rear support member 45 are disposed at predetermined positions, a plurality of sanitary products of the selected type may be set in the housing 50.

When the user takes out the sanitary product 90 from the providing device 1, the user may hold his/her hand over the vicinity of the takeout port 21. As a result, a human sensor built in the providing device 1 senses the user's hand, and a control unit (not illustrated) in the providing device I operates the motor 61 to move the shutter 55 downward. As a result, the takeout port 21 is opened, and the sanitary product 90 located at the lowermost portion in the housing 50 is exposed (see FIG. 3 and the like).

When the shutter 55 is opened, the user can take out one sanitary product from the providing device 1 by pulling out the sanitary product 90 exposed from the takeout port 21 to the front. When the takeout of the sanitary product 90 is completed, the controller operates the motor 61 again to move the shutter 55 upward.

### Internal Configuration of Providing Device

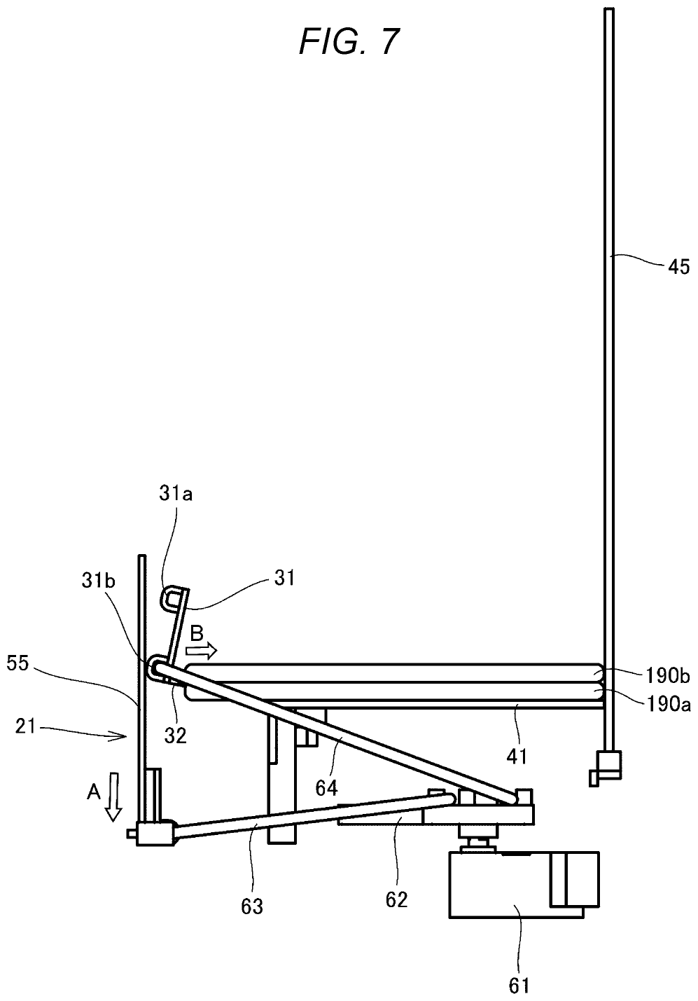

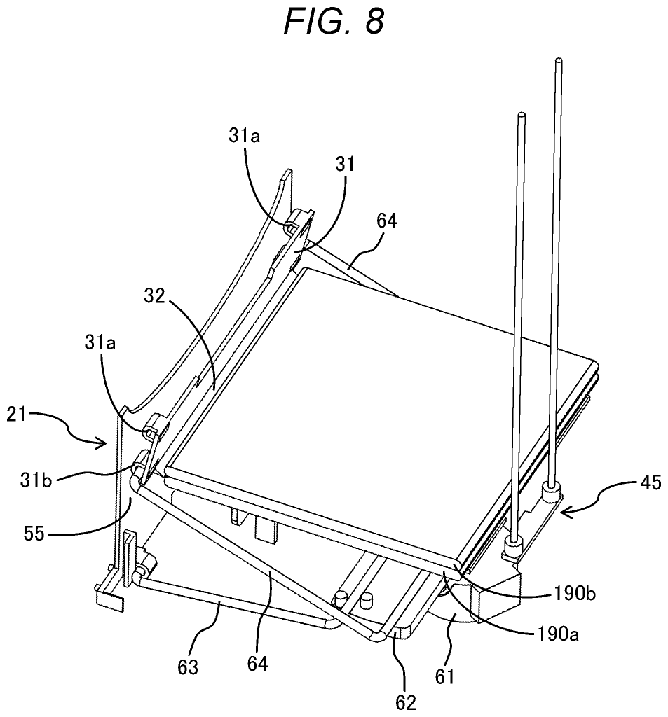

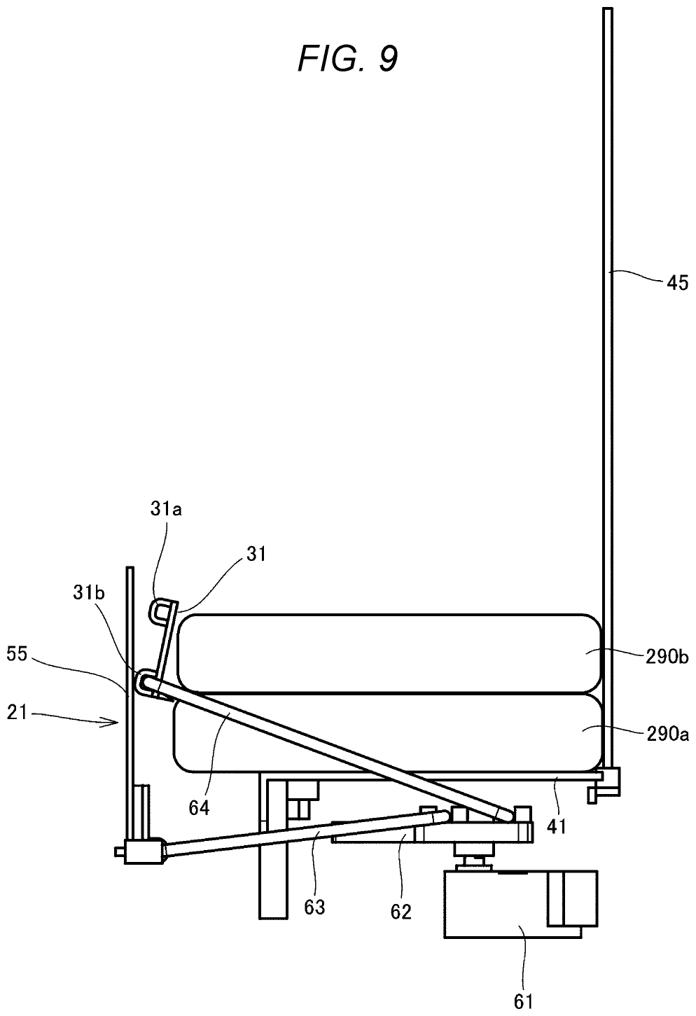

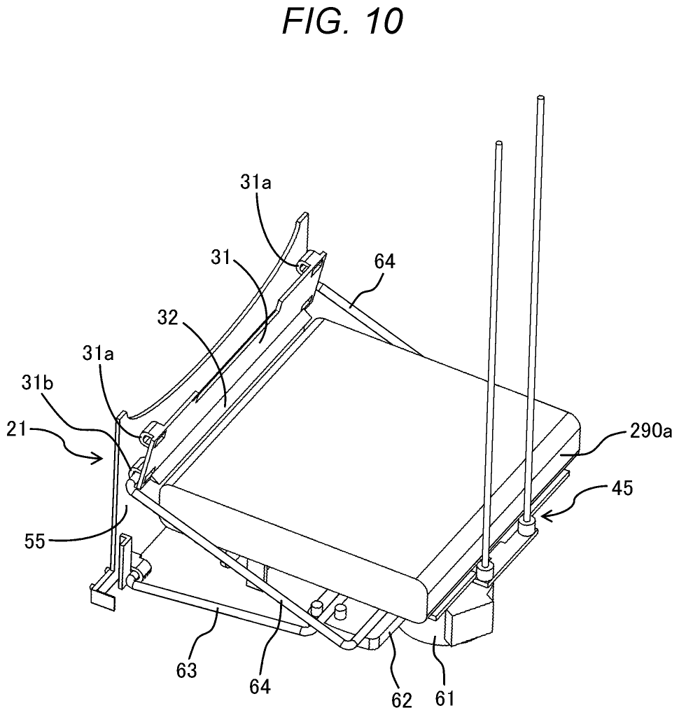

Next, a configuration of the providing device 1 for enabling only one sanitary product 90 to be taken out from the takeout port 21 will be described. FIGS. 7 and 8 illustrate the internal configuration of the providing device 1 in a state where thinner sanitary products 190 (for example, sanitary products having a thickness of less than 1 cm) are accommodated. FIGS. 9 and 10 illustrate the internal configuration of the providing device 1 in a state where thicker sanitary products 290 (for example, sanitary products having a thickness of more than 1 cm) are accommodated.

The housing 50 of the providing device 1 includes a partition member 31, a bottom plate 41, a rear support member 45, a motor 61, a cam 62, a shutter coupling member 63, a partition coupling member 64, and the like.

The partition member 31 is located above the takeout port 21. The partition member 31 has a plate-shaped main body portion, a bearing portion 31a of the rotating shaft, and a coupling portion 31b with the cam.

The bearing portions 31a and 31a are provided on both left and right sides of the upper end portion of the partition member 31, respectively. The bearing portion 31a is a bearing of a rotating shaft for rotating the partition member 31. The rotating shaft is provided, for example, on an inner wall of the side surface portion 50c of the housing 50. The rotating shaft is configured to move in the vertical direction along the inner wall of the housing 50 in accordance with the operation of the cam 62.

The coupling portions 31b and 31b are provided on the left and right sides of the lower end portion of the partition member 31, respectively. The coupling portion 31b pivotally supports the partition coupling member 64 that couples the partition member 31 and the cam 62.

The plurality of stacked sanitary products 90 is placed on the bottom plate 41. As described above, the bottom plate 41 is configured to be movable in the vertical direction according to the thickness of the sanitary product 90 accommodated in the housing 50.

When the sanitary product 190 having a smaller thickness is accommodated in the housing 50, the bottom plate 41 is located at a higher position (see FIG. 7). In addition, when the sanitary product 290 having a larger thickness is accommodated in the housing 50, the bottom plate 41 is located at a lower position (see FIG. 9).

The rear support member 45 extends in the vertical direction at the rear in the housing 50. As described above, the rear support member 45 is configured to be movable in the front-back direction according to the length (depth dimension) in the front-back direction of the sanitary product 90 accommodated in the housing 50.

When the sanitary product 90 having a smaller depth dimension is accommodated in the housing 50, the rear support member 45 is located on a relatively front side. In addition, when the sanitary product 90 having a larger depth dimension is accommodated in the housing 50, the rear support member 45 is located on the more rear side (for example, the vicinity of the back surface portion 50d of the housing 50).

The motor 61 is disposed in the lower part of the housing 50. In the present embodiment, the motor 61 is disposed such that the rotation axis extends along the vertical direction and rotates along the horizontal plane.

The cam 62 is disposed on the motor 61 and is coupled to a rotation shaft of the motor 61. In the present embodiment, the cam 62 has a plate shape and is disposed along a horizontal plane. As a result, the plate-shaped cam 62 rotates along the horizontal plane in accordance with the rotation of the motor 61.

The shutter coupling member 63 is a member that couples the shutter 55 and the cam 62. In the present embodiment, the shutter coupling member 63 is coupled to the lower end portion of the shutter 55. Since the shutter coupling member 63 is provided, the shutter 55 can be moved in the vertical direction in accordance with the rotation operation of the cam 62.

The partition coupling member 64 is a member that couples the partition member 31 and the cam 62. In the present embodiment, the partition coupling member 64 is coupled to the coupling portion 31b provided at the lower end portion of the partition member 31. Since the partition coupling member 64 is provided, the partition member 31 can be moved in the front-rear direction in accordance with the turning operation of the cam 62.

### Operation of Shutter and Partition Member

Next, operations of the shutter 55 and the partition member 31 in the providing device 1 will be described. Here, an operation in a case where a thin sanitary product 190 is accommodated in the providing device I will be described as an example.

FIGS. 7 and 11 to 14 sequentially illustrate the operation of each component in the providing device I until a lowermost sanitary product 190a in the housing 50 is taken out from the takeout port 21 and a sanitary product 190b located immediately above the sanitary product 190a is prepared to be taken out (standby).

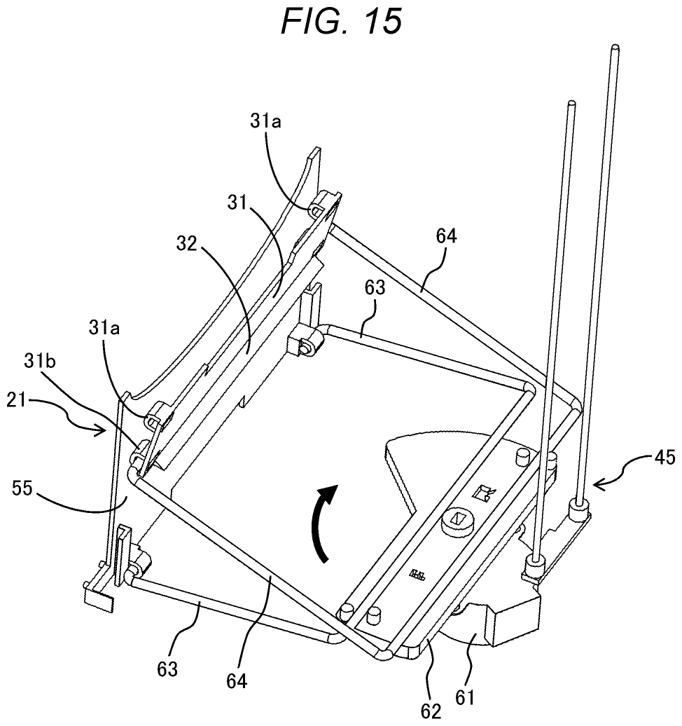

FIGS. 15 to 19 illustrate the positions of the operation mechanisms such as the cam 62 when the providing device 1 is in the states illustrated in FIGS. 7 and 11 to 14, respectively.

FIG. 7 illustrates a state before the providing device 1 starts the operation of providing the sanitary product 190. In this state, the shutter 55 is positioned above and closes the takeout port 21, and the partition member 31 is positioned in front of the sanitary products (for example, the sanitary products 190a, 190b, and the like) positioned on the lower side among the plurality of stacked sanitary products 190, 190, . . . . A folded portion 32 of the partition member 31 is in such a state that the tip portion thereof is positioned between the sanitary product 190a and the sanitary product 190b. That is, the height of the bottom plate 41 is set in advance so as to be in such a state.

At this time, the cam 62 is in a state as illustrated in FIG. 15. This state is defined as a home position of the cam 62.

Thereafter, when the user who desires the sanitary product holds a hand in the vicinity of the takeout port 21 of the providing device 1, the human sensor detects the hand of the user, and a control unit (not illustrated) in the providing device 1 operates the motor 61. As a result, the cam 62 rotates in a direction indicated by an arrow in FIG. 15. When the cam 62 rotates in the direction illustrated in FIG. 15, the shutter 55 moves downward (see an arrow A in FIG. 7), and the coupling portion 31b of the partition member 31 moves backward (see an arrow B in FIG. 7).

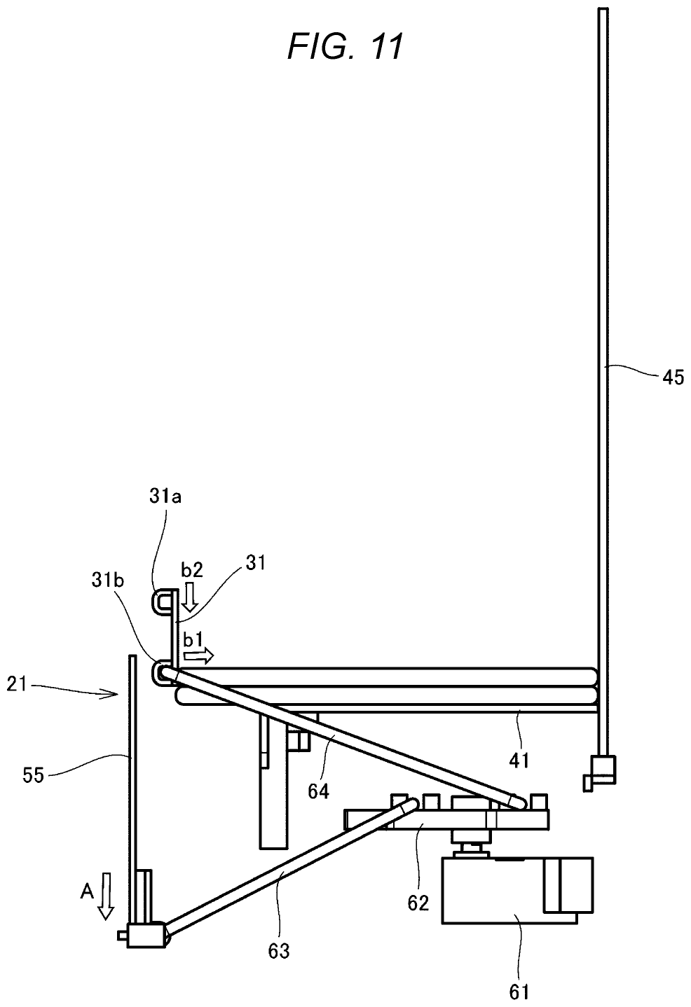

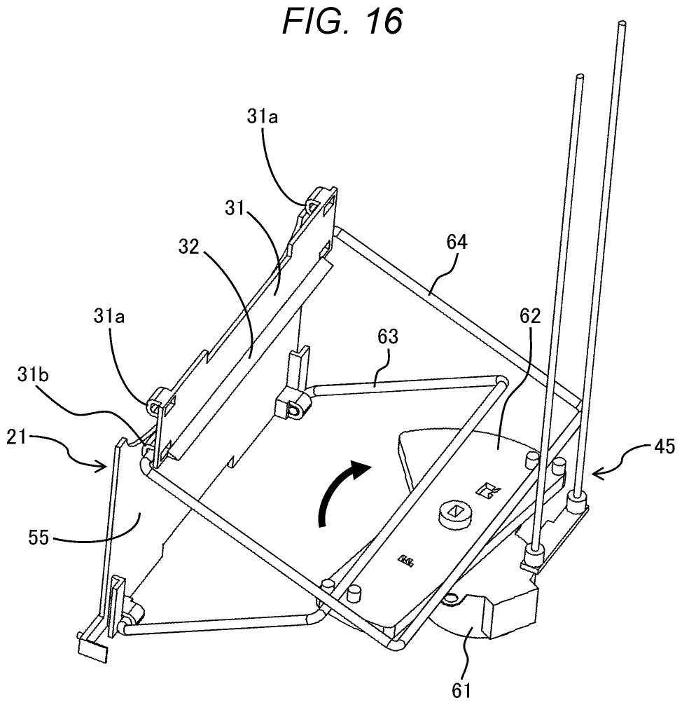

FIGS. 11 and 16 illustrate a state in which the shutter 55 is almost opened. As the cam 62 further rotates in the direction indicated by an arrow in FIG. 16, the shutter 55 further moves downward (see an arrow A in FIG. 11).

When the partition coupling member 64 further moves rearward, the coupling portion 31b of the partition member 31 also moves rearward (see an arrow b1 in FIG. 11). At this time, the rotating shaft inserted into the bearing portion 31a moves downward along a groove provided in the inner wall of the side surface portion 50c of the housing 50 (see an arrow b2 in FIG. 11). As a result, the rotating shaft inserted into the bearing portion 31a moves downward while rotating rearward.

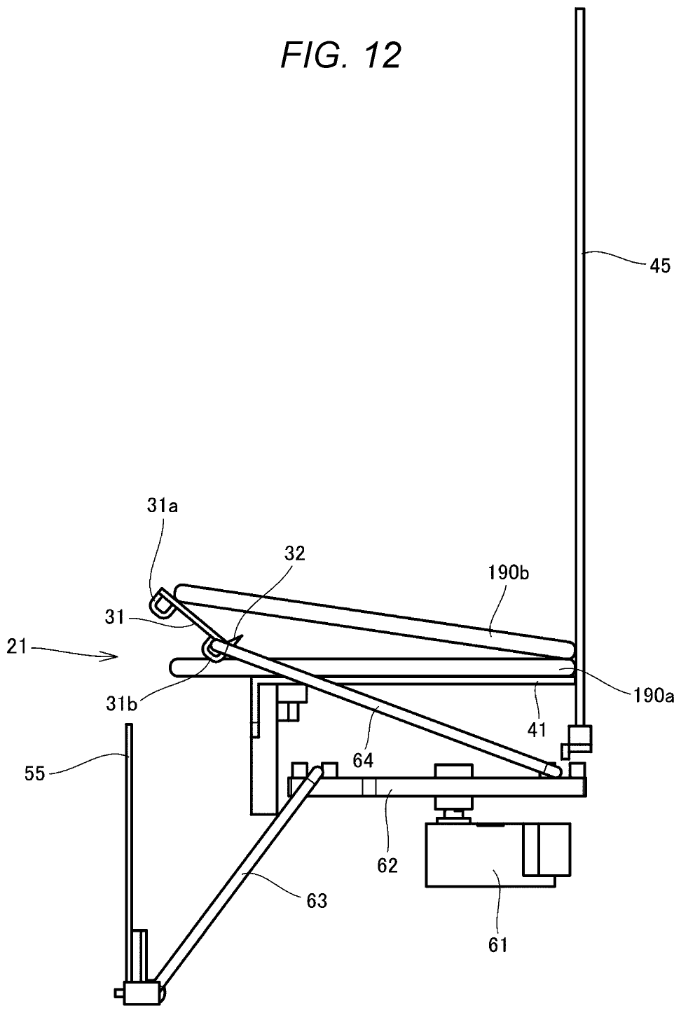

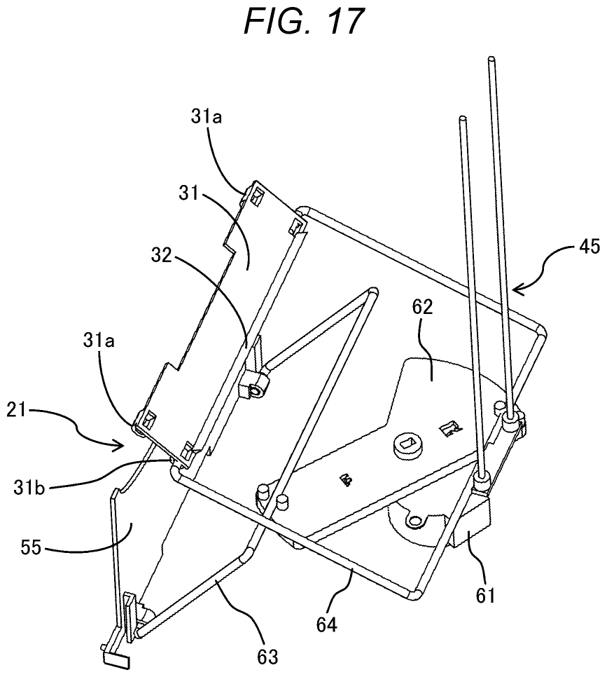

By the partition member 31 operating as described above, the folded portion 32 located at the lower end portion of the partition member 31 is inserted between the sanitary product 190a and the sanitary product 190b. Then, when the rotation of the cam 62 further progresses to a state as illustrated in FIG. 17, the shutter 55 is fully opened. The positions of the shutter 55 and the partition member 31 at this time are illustrated in FIG. 12.

As illustrated in FIG. 12, when the shutter 55 is fully opened, most of the partition member 31 is inserted between the sanitary product 190a and the sanitary product 190b. As a result, the sanitary product 190a located at the lowermost portion is exposed from the takeout port 21 and can be taken out by the user. On the other hand, the sanitary product 190b located immediately above the sanitary product 190a is hidden by the partition member 31 and is not exposed from the takeout port 21 (see FIG. 4).

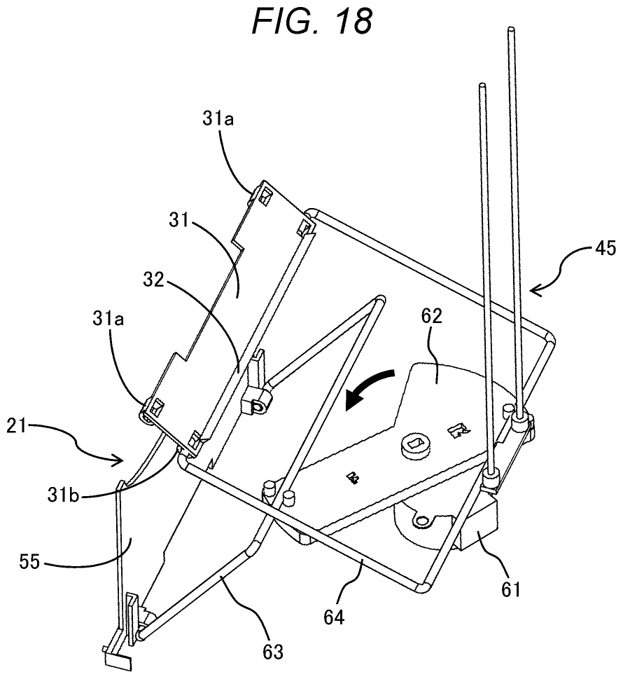

Thereafter, when the user takes out the sanitary product 190a from the providing device 1, the control unit in the providing device 1 operates the motor 61 again to rotate in a direction opposite to the previous direction. As a result, the cam 62 rotates in a direction indicated by an arrow in FIG. 18. Here, the rotating operation of the motor 61 in the reverse direction may be started when a predetermined time has elapsed since the shutter 55 was opened, or may be started when the sensor detects that the sanitary product 190a has been completely removed.

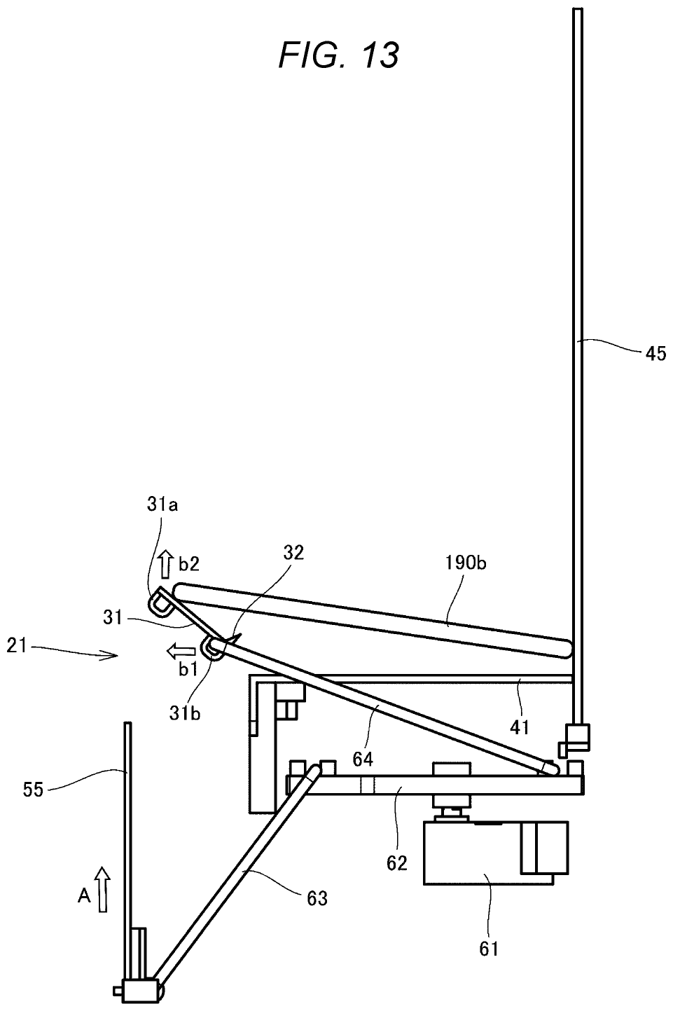

When the cam 62 rotates in the arrow direction in FIG. 18, the shutter 55 moves upward (that is, the direction in which the takeout port 21 is closed) (see an arrow A in FIG. 13).

When the cam 62 rotates in the arrow direction in FIG. 18, the coupling portion 31b of the partition member 31 moves forward (see an arrow b1 in FIG. 13). At this time, the rotating shaft inserted into the bearing portion 31a moves upward along a groove provided in the inner wall of the side surface portion 50c of the housing 50 (see an arrow b2 in FIG. 13). As a result, the rotating shaft inserted into the bearing portion 31a moves upward while rotating forward.

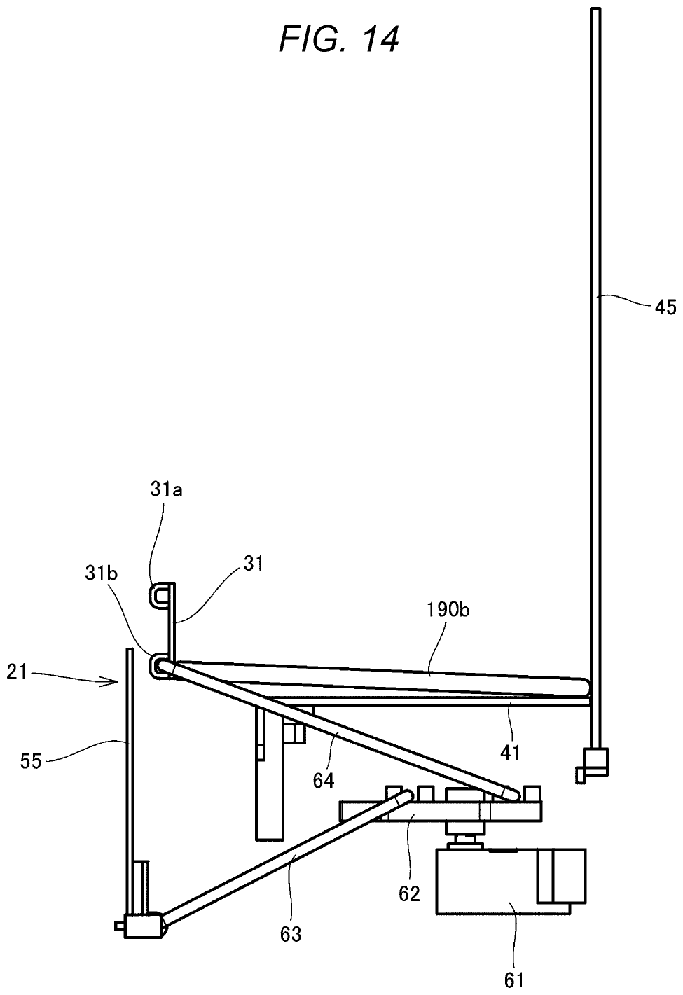

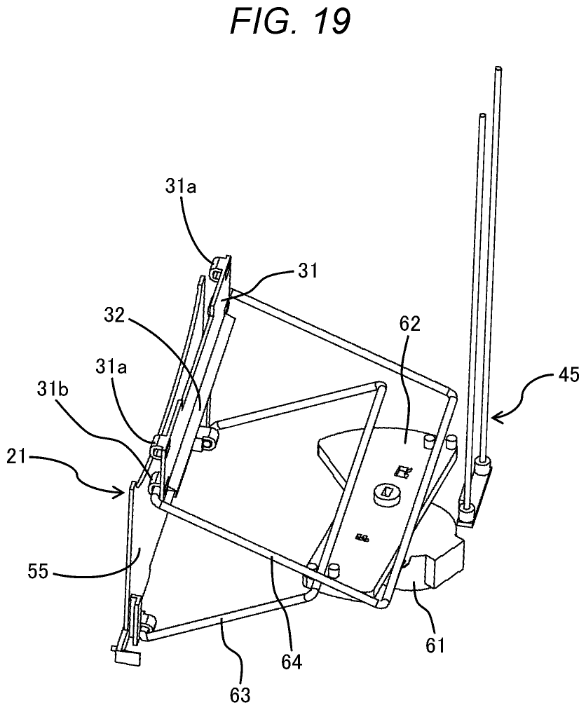

Thereafter, the cam 62 returns to the home position state as illustrated in FIG. 15 through the state illustrated in FIG. 19. Accordingly, the takeout port 21 is completely closed by the shutter 55. Then, as the partition member 31 inserted under the sanitary product 190b is pulled out, the sanitary product 190b that has been located immediately above the sanitary product 190a until now falls on the upper surface of the bottom plate 41 (see FIG. 14).

Thereafter, the sanitary product 190b is placed at the position of the sanitary product 190a shown in FIG. 7, and the motor 61 stops operating. In this state, preparation for the next operation of providing the sanitary product 190 to the user is made.

As described above, in the providing device 1 according to the present embodiment, the partition member 31 moves backward while lowering at the timing when the shutter 55 is opened, and is inserted between the sanitary product 190a and the sanitary product 190b. As a result, the sanitary product 190a located at the lowermost portion is exposed from the takeout port 21 and can be taken out by the user, while the other sanitary products 190 are hidden by the partition member 31 or the like and cannot be taken out from the takeout port 21.

In the providing device 1 according to the present embodiment, since the folded portion 32 is provided, the insertion operation of the partition member 31 between the sanitary product 190a and the sanitary product 190b can be more reliably performed.

In the above description, the operation of the shutter 55 and the partition member 31 in the case of providing the thin sanitary product 190 has been described. However, also in a case where the thick sanitary product 290 is accommodated in the providing device 1, the shutter 55 and the partition member 31 operate substantially in the same manner as described above.

In a case where a thick sanitary product 290 is accommodated in the providing device 1, by appropriately changing the positions of the bottom plate 41 and the rear support member 45, similarly to the case of the thin sanitary product 190, one sanitary product can be appropriately provided to a user who desires the sanitary product. Therefore, according to the providing device 1 according to the present embodiment, it is possible to appropriately provide one hygiene product regardless of the size of the accommodated hygiene product.

As described above, in the providing device 1 according to the present embodiment, the partition member 31 that operates in conjunction with the opening/closing operation of the shutter 55 moves in the front-rear direction. For example, when the shutter 55 is opened, the partition member 31 moves backward and is inserted from the front between one sanitary product 90a located at the lowermost portion in the housing 50 and a sanitary product located at the second lowermost portion.

Then, the sanitary product 190a located at the lowermost portion is exposed from the takeout port 21 and can be taken out by the user, while the other sanitary products 190 are hidden by the partition member 31 and the like and cannot be taken out from the takeout port 21. As a result, it is possible to appropriately provide one sanitary product without finely adjusting the size of the opening of the takeout port 21 such as adjusting the opening degree of the shutter 55. Therefore, one sanitary product can be appropriately provided to a user who desires the sanitary product.

### SUMMARY

As described above, a hygiene product providing device (for example, the hygiene product providing device 1) according to one aspect of the present disclosure includes: a housing (for example, the housing 50) that accommodates a plurality of hygiene products (for example, any one of the sanitary products 90, 190, and 290) in a stacked manner; a takeout port (for example, the takeout port 21) of the hygiene product disposed below a front surface portion (for example, the front surface portion 50a) of the housing; and a shutter (for example, the shutter 55) that opens and closes the takeout port. When the shutter is opened, one hygiene product (for example, the sanitary product 190a) located at a lowermost portion in the housing is exposed from the takeout port, and a hygiene product (for example, the sanitary product 190b) located above the one hygiene product located at the lowermost portion is not exposed from the takeout port.

According to the above configuration, it is possible to appropriately provide one hygiene product to the user when the shutter is opened.

In the hygiene product providing device (for example, the hygiene product providing device 1) according to one aspect of the present disclosure, when the shutter (for example, the shutter 55) is opened, the plate member (for example, the partition member 31) is inserted from the front between the one hygiene product (for example, the sanitary product 190a) located at the lowermost portion and the hygiene product (for example, the sanitary product 190b) located at the second lowermost portion, so that the hygiene product located above the one hygiene product located at the lowermost portion may not be exposed from the takeout port.

According to the above configuration, the plate-shaped member operates as described above in conjunction with the opening/closing operation of the shutter, whereby one sanitary product can be appropriately provided to the user who desires the sanitary product. In addition, it is possible to set a state in which the user cannot touch the sanitary products other than the sanitary product located at the lowermost portion.

In the hygiene product providing device (for example, the hygiene product providing device 1) according to one aspect of the present disclosure, the folded portion (for example, the folded portion 32) may be provided at the lower end portion of the plate-shaped member (for example, the partition member 31). Since the folded portion is provided, the partition member can be more reliably and smoothly inserted between the lowermost sanitary product and the sanitary product located immediately above the lowermost sanitary product.

The hygiene product providing device (for example, the hygiene product providing device 1) according to one aspect of the present disclosure may further include a bottom plate (for example, the bottom plate 41) which is disposed in the housing and on which the hygiene product is placed, and the bottom plate may be configured to be vertically movable in accordance with a size of the hygiene product.

A hygiene product providing device (for example, the hygiene product providing device 1) according to one aspect of the present disclosure further includes a rear support member (for example, the rear support member 45) which is located on a rear side of the hygiene product housed in the housing and supports the hygiene product, and the rear support member may be configured to be movable back and forth in accordance with a size of the hygiene product.

It should be understood that the embodiment disclosed herein is illustrative in all respects and not restrictive. The scope of the present disclosure is defined not by the above description but by the claims, and it is intended that meanings equivalent to the claims and all modifications within the scope are included.

What is claimed is:

1. A hygiene product providing device comprising:
a housing configured to accommodate a plurality of hygiene products in a stacked manner;
a takeout port of the hygiene product disposed in a lower portion of a front surface portion of the housing; and
a shutter configured to open and close the takeout port,
wherein when the shutter is opened, one hygiene product located at a lowermost portion in the housing is exposed from the takeout port, and a hygiene product located higher than the one hygiene product located at the lowermost portion is not exposed from the takeout port.

2. The hygiene product providing device according to claim 1, wherein when the shutter is opened, a plate-shaped member is inserted from a front between the one hygiene product located at the lowermost portion and a hygiene product located at a second lowermost position, so that a hygiene product located above the one hygiene product located at the lowermost portion is not exposed from the takeout port.

3. The hygiene product providing device according to claim 2, wherein a lower end portion of the plate-shaped member moves in a front-rear direction in conjunction with an opening/closing operation of the shutter.

4. The hygiene product providing device according to claim 3, wherein a folded portion is provided at a lower end portion of the plate-shaped member.

5. The hygiene product providing device according to claim 1, further comprising a bottom plate that is disposed in the housing and on which the hygiene product is placed,
wherein the bottom plate is configured to be vertically movable in accordance with a size of the hygiene product.

6. The hygiene product providing device according to claim 1, further comprising a rear support member that is located on a rear side of the hygiene product accommodated in the housing and supports the hygiene product,
wherein the rear support member is configured to be movable back and forth in accordance with a size of the hygiene product.
